(19) United States
(12) Patent Application Publication
(10) Pub. No.: US 2004/0203123 A1
(43) Pub. Date: Oct. 14, 2004

(54) Method for transforming Amycolatopsis sp. DSM 9991 and DSM 9992

(75) Inventors: Jurgen Rabenhorst, Hoxter (DE); Alexander Steinbuchel, Altenberge (DE); Horst Priefert, Ostbevern (DE); Sandra Achterhold, Saerbeck (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG.

(21) Appl. No.: 10/796,306

(22) Filed: Mar. 10, 2004

(30) Foreign Application Priority Data
Aug. 29, 2002 (WO) PCT/EP02/09619
Sep. 10, 2001 (DE) 101 44 308.0

(51) Int. Cl. C12P 7/24; C12N 9/26; C12N 15/74

(52) U.S. Cl. 435/147; 435/471; 435/252.3

(57) ABSTRACT

The invention relates to a method for the transformation of Amycolatopsis sp. DSM 9991 and DSM 9992 and the use of the strains transformed in this way for the preparation of vanillin, preferably for the preparation of vanillin from ferulic acid.

### FIELD OF THE INVENTION

The invention relates to a method for the transformation of Amycolatopsis sp. DSM 9991 or DSM 9992 and the use of the strains transformed in this way for the preparation of vanillin, preferably for the preparation of vanillin from ferulic acid.

### BACKGROUND OF THE INVENTION

Vanillin is an important flavouring agent widely used in the food and luxury food industries. It is prepared by a chemical route, mainly from lignin contained in sulphite spent liquors and also by oxidation of eugenol or isoeugenol. However, vanillin prepared by a chemical route has the disadvantage that it is not a natural substance as defined by some food laws and, therefore, may not be designated as a natural flavouring agent.

Hitherto, the natural flavouring agent vanillin has been obtainable only by extraction from vanilla pods, but the vanillin obtained in this way is very expensive. Various other methods for the preparation of natural vanillin using various microorganisms and enzymes are already known (see, for example, EP 405 197 A, EP 453 368 A and EP 542 348 A), but up to now are unsuitable for an industrial preparation because of the low yields and concentrations of vanillin.

U.S. Pat. No. 6,133,003 (equivalent to EP 761 817) describes a method for the fermentative preparation of natural vanillin from ferulic acid in which two strains of the genus Amycolatopsis (Pseudonorcardiaceae family) which have been deposited with the Deutsche Sammlung für Mikroorganismen und Zellkulturen GmbH in Braunschweig under numbers DSM 9991 and DSM 9992 (date of first deposition: 2 May 1995) were used. With the aid of these strains and of the method described in U.S. Pat. No. 6,133,003 (EP 761 817 A), it was possible to obtain natural vanillin in good yields and high concentrations in an economic manner. The disclosure of this patent is herein incorporated by reference.

Briefly described, U.S. Pat. No. 6,133,003 describes a process in which vanillin is made by subjecting ferulic acid to Amycolatopsis sp. DSM 9992, or mutant thereof or an isolated enzyme thereof which converts ferulic acid to vanillin, for a period of time sufficient to convert said ferulic acid to vanillin, and recovering the vanillin thus formed. Natural ferulic acid is preferred as the starting material and can be obtained, inter alia, from natural eugenol by conversion with Pseudomonas sp. DSM 7062 or DSM 7063 (see, DE-A 4 227 076).

The organism is cultured in a conventional culture medium in a conventional manner for the culturing of microorganisms. The substrate can be added at the beginning of the incubation, during or after completion of growth, all at once or distributed over a relatively long period. The amount of ferulic acid is advantageously of a magnitude such that the concentration of the compound in the culture broth does not exceed 80 g/l, preferably 15 g/l. The course of the reaction can be followed by determining the starting material and the product in the culture broth by high-pressure liquid chromatography. After the optimum amount of vanillin has formed, this is isolated from the culture broth by known physical methods such as extraction, distillation or chromatography. The crude product thus obtained can be purified by further steps.

As described in U.S. Pat. No. 6,133,003, the microorganism is cultured in synthetic, semisynthetic or complex culture media. These culture media contain carbon sources, nitrogen sources, inorganic salts and, if appropriate, trace elements and vitamins. Carbon sources which can be used include sugars such as glucose, sugar alcohols such as glycerol or mannitol, organic acids such as citric acid, or complex mixtures such as malt extract, yeast extract, casein or casein hydrolysate. Examples of suitable nitrogen sources are inorganic nitrogen sources such as nitrates and ammonium salts, and organic nitrogen sources such as yeast extract, soya bean meal, cotton seed meal, casein, casein hydrolysate, wheat gluten and corn steep liquor. Inorganic salts which can be used include sulphates, nitrates, chlorides, carbonates and phosphates of sodium, potassium, magnesium, calcium, zinc and iron.

The culture temperature is preferably in the range from 10 to 55° C., particularly preferably in the range from 35 to 45° C. The pH of the medium is preferably 3 to 9, in particular 4 to 8. The microorganisms can be cultured either in suitable shaking apparatuses or in fermenters equipped with a stirrer device. Care must be taken to ensure adequate aeration in culturing. The microorganisms can be cultured batchwise, semicontinuously or continuously. The culture time until a maximum amount of product has been achieved is between 4 and 120 hours after inoculation of the culture. To protect the microorganisms from the toxic activity of the substances used or formed, it can be advantageous to add adsorbents to the culture media, e.g. activated carbon or adsorber resins such as Amberlite® XAD-2, Amberlite® XAD-7, XAD-16, Lewatit® OC 1062 or OC 1064.

It is known that the use of genetically modified microorganisms in fermentative methods often leads to improved yields or concentrations of desired substances. In order to obtain such genetically modified microorganisms, it is necessary to know a suitable method for transformation of these microorganisms.

Methods for the transformation of various Amycolatopsis species are known. A method for the protoplast transformation of Amycolatopsis orientalis is described in P. Matsushima et al. J. Bacteriol. 169, 1987, 2298-2300. A disadvantage of this method is that it cannot be applied to the transformation of Amycolatopsis sp. DSM 9991 and DSM 9992 (see examples). An electroporation method for the transformation of Amycolatopsis mediterranei is described in R. Lal et al. J. Antibiotics 51, 1998, 161 - 169. A disadvantage is that, when this method was applied to Amycolatopsis sp. DSM 9991 and DSM 9992, only low transformation rates could be achieved (see examples).

The direct mycelium transformation of Amycolatopsis mediterranei is described in J. Madon et al. J. Bacteriol. 173, 1991, 6325-6331. A modification of this method for the direct mycelium transformation of Amycolatopsis methanolica is described in J. W. Vrijbloed et at. (sic) Plasmid 34, 1995, 96-104. Disadvantages of this method are the low rates of transformation which result because of the long incubation period of the mycelium of approximately 40 hours.

There was therefore a need to find a transformation method that is suitable for Amycolatopsis sp. DSM 9991 or DSM 9992, with which high rates of transformation can be achieved.

### SUMMARY OF THE INVENTION

Surprisingly, the invention provides a method for the transformation of Amycolatopsis sp. DSM 9991 or DSM 9992 by

(1) culturing of Amycolatopsis sp. DSM 9991- or DSM 9992 mycelia in a culture medium and

(2) bringing this culture into contact with a mixture containing

(a) 0.25 to 10 μg/ml DNA to be transformed

(b) 0.4 to 0.7 M CsCl

(c) 0 to 9 mM MgCl₂

(d) 30 to 50% [m/V] polyethylene glycol 1000 and

(e) 10 to 50 μg/ml DNA which differs from (a),

the mycelium being brought into contact with the said mixture 4.5 to 9 hours after stationary mycelia cells are formed.

### DETAILED DESCRIPTION

Step (1) of the method according to the invention relates to the culturing of Amycolatopsis sp. DSM 9991 or DSM 9992 mycelia in a culture medium. Suitable culture media are those such as are known in the literature for the culturing of mycelia of the species Amycolatopsis. Particularly suitable culture media are complex media such as, for example, YMG medium (0.4% [m/V] yeast extract, 1% [m/V] malt extract, 0.4% [m/V] glucose, pH 7.2), TYN medium (0.25% [m/V] yeast extract, 1% [m/V] tryptone, 0.5% [m/V] NaCl, pH 7.2) or TSB medium (1.7% [m/V] tryptone, 0.3% [m/V] Soytone, 0.25% [m/V] glucose, 0.5% [m/V] NaCi, 0.25% [m/V] K₂HPO₄ pH 7.3). The culturing is preferably carried out at temperatures within the range of about 30 to 48° C., particularly preferred is a temperature within the range of 39 to 42° C.

Preferably, the procedure in Step (1) of the method according to the invention is that a preliminary culture of Amycolatopsis sp. DSM 9991 or DSM 9992 mycelia is cultured in a suitable culture medium. Preferably, the preliminary culture is cultured at the same temperature and in the same culture medium as the actual culture. To prepare a culture of Amycolatopsis sp. DSM 9991 or DSM 9992, a portion of the preliminary culture is used for inoculation, preferably after 16 to 24 hours, particularly after 18 to 22 hours, and most preferably after 19 to 20 hours.

The growth of Amycolatopsis sp. DSM 9991 or DSM 9992 mycelia in the culture medium can, for example, be determined with the aid of spectrometric methods. Preferably, the growth is determined via the optical density of the culture. According to the method according to the invention the transformation of Amycolatopsis sp. DSM 9991 or DSM 9992 mycelia takes place 4.5 to 9 hours after stationary phase mycelia cells are formed. It has been found, surprisingly, that within this time window particularly high rates of transformation can be achieved, which are distinctly higher than when the transformation methods described in the state of the art are used. Of particularly interest, the transformation takes place 5 to 8.5 hours after entry into the stationary phase and especially after 6.5 to 7.5 hours from the formation of stationary mycelia cells.

For transformation, the mycelium culture of Amycolatopsis sp. DSM 9991 or DSM 9992 is brought into contact with a mixture containing:

(a) 0.25 to 10 μg/ml DNA to be transformed

(b) 0.4 to 0.7 M CsCl

(c) 0 to 9 mM MgCl₂

(d) 30 to 50% [m/V] polyethylene glycol having an average molecular weight of 1000, and

(e) 10 to 50 μg/ml DNA which differs from (a).

This mixture is referred to as the “transformation mixture” below.

For transformation, an aliquot of the mycelium culture is preferably centrifuged off, washed and then resuspended in the wash solution or in a suitable buffer, preferably in TE buffer. suitable buffers can be used as wash solution; TRIS-EDTA buffer (10 mM TRIS-HCl, pH 8.0, 1 mM EDTA) is preferably used. During the resuspension the mycelium culture is preferably diluted to an optical density of 25 to 160 (at 400 nm), particularly preferentially to an optical density of 30 to 100 (at 400 nm), and very particularly preferentially to an optical density of 40 to 60 (at 400 nm).

The mycelium culture is preferably brought into contact by mixing with the abovementioned transformation mixture. The mixture thus obtained is incubated at a temperature of preferably 30 to 46° C., particularly preferentially at 37 to 40° C., preferably for 20 to 60 minutes and particularly preferentially for 30 to 40 minutes. It has proved advantageous to wash the mycelia after incubation. The wash liquids used are preferably isotonic media, particularly preferentially S27M medium (7.32% [m/V] D-mannitol, 0.5% [m/V] peptone, 0.3% [m/V] yeast extract, 0.2% [m/V] CaCO₃). Washing can be carried out once or several times.

The transformation mixture used in the method according to the invention contains the abovementioned compounds (a)-(e).

The transformation mixture used in the method according to the invention contains 0.25 to 10 μg/ml DNA to be transformed, preferably 1 to 7.5 μg/ml, particularly preferentially 2 to 6 μg/ml. The DNA to be transformed can be in the form of single-strand or double-strand DNA; preferably DNA is used in the form of double-strand circular DNA (plasmids). The plasmids preferably used in the transformation contain the following components in particular: at least one source of replication, which enables the efficient replication of the plasmid in Amycolatopsis sp. DSM 9991 or 9992. Preferably, the plasmid additionally contains a replication source that enables the efficient replication of the plasmid in a cell that is suitable for single production and isolation of the plasmid (for example Escherichia coli). The plasmid furthermore preferably contains a resistance gene, which enables the selection of Amycolatopsis sp. DSM 9991 or 9992 cell clones, which contain the plasmid, preferably a kanamycin resistance gene, but not an erythromycin or thiostrepton resistance gene, since Amycolatopsis sp. DSM 9991 or 9992 have (sic) an inherent erythromycin and thiostrepton resistance, respectively. Preferably, the plasmid contains restriction interfaces for the incorporation of foreign DNA fragments. Preferably, the DNA to be transformed is a DNA which has a low degree of methylation. This can be achieved in that the DNA to be transformed is isolated from an organism that is not able to modify DNA or is able to do so only to a slight extent. These organisms are known to those skilled in the art; for example, various Escherichia coli strains, such as, for example, E. coli ET12567 (dam, dcm, hsd) or E. coli JM110 (dam, dcm) or Amycolatopsis sp. DSM 9991 or 9992 itself can be employed.

The transformation mixture used in the method according to the invention contains 0.4 to 0.7 M CsCl, preferably 0.5 to 0.675 M CsCl and particularly preferentially 0.575 to 0.625 M CsCl.

The transformation mixture used in the method according to the invention contains 0 to 9 mM MgCl₂, preferably 2.5 to 7.5 mM MgCl₂ and particularly preferentially 3.5 to 5.5 mM MgCl₂.

The transformation mixture used in the method according to the invention contains 30 to 50% [m/V] polyethylene glycol having an average molecular weight of 1000 (hereinafter designated “PEG 1000”), preferably 31 to 40% [m/V] PEG 1000 and particularly preferentially 32 to 35% [m/V] PEG 1000. The use of PEG 1000 is advantageous since it was possible to achieve only low rates of transformation when a PEG which has a higher or a lower molecular weight is used.

The transformation mixture used in the method according to the invention contains 10 to 50 μg/ml DNA which differs from (a), preferably 12 to 30 μg/ml and particularly preferentially 15 to 25 μg/ml. The presence of (e) in the transformation mixture makes it possible to keep the concentration of component (a) low. Calf thymus DNA is preferably used for this purpose and ultrasound treated calf thymus DNA is particularly preferentially used.

In a preferred embodiment the transformation mixture used in the method according to the invention contains:

1(a) 0.25 to 10 μg/ml DNA to be transformed

(b) 0.575 to 0.625 M CsCl

(c) 2.5 to 7.5 mM MgCl2

(d) 32 to 35% [m/V] polyethylene glycol 1000 and

(e) 12 to 30 μg/ml DNA which differs from (a).

In a particularly preferred embodiment the transformation mixture used in the method according to the invention contains:

(a) 2 to 6 μg/ml DNA to be transformed

(b) 0.575 to 0.625 M CsCl

(c) 3.5 to 5.5 mM MgCl₂

(d) 32 to 35% [m/V] polyethylene glycol 1000 und

(e) 15 to 25 μg/ml DNA which differs from (a).

After the transformation the mycelium culture is preferably mixed with an R2L agarose solution, as described in J. Madon et al. J. Bacteriol. 173, 1991, 6325 - 6331, the R2L solution preferably being temperature-controlled to 37 to 46° C. and particularly preferentially to 40 to 42° C. The mixture is then applied to agar plates, preferably S27M agar plates. The incubation time is preferably 14 to 22 hours, particularly preferentially 16 to 20 hours; the incubation temperature is preferably 30° C. After incubation, selection takes place preferably by covering the plates with a layer of soft agar containing antibiotics (0.5% [m/V] agar), preferably S27M soft agar, and subsequent incubation at, preferably, 30 to 37° C. for preferably 5 to 10 days.

By means of the method according to the invention it is possible to transform Amycolatopsis sp. DSM 9991 and DSM 9992 in a simple manner, high rates of transformation being obtained.

### EXAMPLES

### Example 1

Construction and Isolation of the Plasmid used for the Transformation

The vector pRL60 (Lal et al., J. Antibiotics 51, 1998, 161 - 169) was used for construction of a plasmid suitable for the transformation. This vector, which is 10.2 kbp in size, contains a replication source (pA-rep) for various Amycolatopsis mediterranei strains, a replication source (pBR-ori) for Escherichia coli, a kanamycin resistance gene, an erythromycin resistance gene and an α-amylase marker gene. The plasmid was subjected to restriction digestion with EcoRI and separated in an agarose gel, and a fragment 6 kbp in size, which contained the kanamycin resistance gene, pA-rep and pBR-ori, was isolated therefrom. The fragment was religated and the plasmid pRLE6 (5843 bp) thus obtained was transformed in competent E. coli ET12567 cells and isolated therefrom. Restriction digestion, separation in agarose gel, isolation of the desired DNA fragment, religation, transformation in E. coli and isolation of the plasmid were carried out in accordance with the standard protocols customary in molecular biology (see, for example, J. Sambrook et al., Molecular cloning: a laboratory manual, 2^{nd} ed. Cold Spring Harbor Laboratory, Cold Spring Harbor, N.Y.).

### Example 2 (not according to the invention)

Protoplast Transformation of Amycolatopsis sp. DSM 9992

(modified according to Hopwood et al 1985, Genetic manipulation of Streptomyces—a labor (sic) manual, The John Innes Foundation, Norwich, England.)

The following methods, media and buffers were used:

Buffer for Protoplast Formation (P buffer) for Streptomycetes

Make up 103 g sucrose, 0.25 g MgCl₂×6H₂O, 2.02 g K₂SO₄, and 2 ml trace element solution 2 (described in Hopwood et al 1985, “Genetic manipulation of Streptomyces—a manual”, The John Innes Foundation, Norwich, England) to 790 ml with water and autoclave. After autoclaving, the following sterilised solutions are added separately thereto: 100 ml TES (5.73% [m/V], adjusted to pH 7.2 with NaOH), 100 ml CaCl₂×2H₂O (3.68% [m/V]) and 10 ml K₂HPO₄ (0.5% [m/V]).

R3 Regeneration Medium for Protoplasts

Make up 103 g sucrose, 10 g glucose (monohydrate), 0.5 g KCl, 4 g peptone, 4 g yeast extract, 8.1 g MgCl₂×6H₂O, 2.2 g CaCl₂×2H₂O to 880 ml with water, add 18 g Bacto agar and autoclave. After autoclaving, the following sterilised solutions are added separately thereto: 100 ml TES (5.73% [m/V], adjusted to pH 7.2 with NaOH), 20 ml K₂HPO₄ (1% [mN/V]).

Transformation Buffer for Protoplasts

All solutions are prepared and autoclaved separately and then mixed as indicated: 25 ml sucrose (10.3% [m/V]), 75 ml water_{2x dist}, 0.2 ml trace element solution 2 (described in Hopwood et al. 1985, “Genetic manipulation of Streptomyces—a manual”, The John Innes Foundation, Norwich, England) and 1 ml K₂SO₄ (2.5% [m/V]).

From the mixture, 9.3 ml are taken and the following solutions added thereto: 0.2 ml 5 M CaCl₂ and 0.5 ml 1 M TRIS maleate, pH 8. Before use, 3 parts of the transformation buffer are mixed with 1 part [m/V] sterile PEG 1550.

Determination of the Protoplast Titre and of the Regeneration Rate

To determine the regeneration rate dilution series (10⁻¹-10⁻⁶) were prepared in P buffer and in H₂O_{2x dist} (+0.01% [m/V] sodium dodecylsulphate) and 100 μl of each of the dilution steps was applied with 3 ml “Top agar” (38° C., P buffer to which 0.4% [m/V] low melting point agarose, Sigma, has been added) to R3 regeneration medium for protoplasts. The plates were dried for 15-20 min under the sterile workbench before they were incubated at 37° C. After 4-6 days the number of colonies per plate was counted.

(a) Protoplast Isolation

Culturing of the mycelium was carried out for 15 h in 50 ml YMG medium with 5% [V/V] PEG 6000 in 300 ml baffle flasks at 37° C. and 150 rpm. The cell material was centrifuged off at 3 000 rpm for 15 min under sterile conditions and then washed twice with, in each case, 15 ml of a sterile 10.3% [m/V] sucrose solution. The mycelium pellet centrifuged off was subjected to a lysozyme treatment in order to achieve (partial) digestion of murein sacculus. For this purpose the pellet was resuspended in 4 ml lysozyme solution (2 mg/ml dissolved in sterile P buffer, see below) and incubated at 30° C. with gentle shaking (120 rpm). The progress of the protoplast formation was followed microscopically throughout the entire period. Protoplasts were already discemable after 15-30 min. After 2 to 2 ¼ h the suspension was sucked up and extracted 3 times with a sterile 5 ml pipette in order to achieve better protoplast liberation. The suspension was then incubated for a further 15-30 min.

After adding 5 ml P buffer separation of the protoplasts from the mycelium residues was carried out by differential centrifuging. After centrifuging for 10 min at 1 100 rpm (Megafuge 1.0R)—corresponding to approx. 200 g—the supernatant containing the protoplasts was decanted off from the mycelium residues and centrifuged again. This step was carried out at 3 400 rpm (approx. 2 000 g) for 10 min. The protoplasts thus obtained were resuspended in the residual liquid. To check the protoplast formation, 50 μl P buffer and, respectively, 50 μl H₂O_{2x dist} (+0.01% [m/V] sodium dodecylsulphate) was added to 50 μl suspension in each case and the mixtures viewed under the microscope.)

3 washing steps followed. For this purpose 10 ml sterile P buffer were added to the “creamy” protoplast suspension and, after shaking gently, this was followed by centrifuging for 10 min at 3 400 rpm and 4° C. Finally, the protoplasts were resuspended [lacuna] P buffer. The buffer volume (approx. 1-3 ml) was so chosen that a titre of approx. 10⁹-10¹⁰ protoplasts per ml was achieved. The protoplasts treated in this way could be stored at −70° C. for a prolonged period until further use. The cell aliquots were slowly frozen on ice at −70° C.; the protoplasts were thawed under lukewarm water.

(b) Protoplast Transformation

For the transformation, 100 μl of the protoplast suspension (see above for preparation) were centrifuged briefly in the table-top centrifuge for pelletising. The supernatant liquor was decanted off and the pellet resuspended in the “residual liquid” (by tapping with the fingers). Plasmid DNA (in TE buffer; max. 20 μl) was added to this protoplast suspension. A batch without plasmid DNA was carried out in parallel as a control. Immediately thereafter, 0.5 ml transformation buffer to which 25% [V/V] PEG 1 550 had been added, were (sic) added and sucked up and extracted twice using the pipette and 50 μl aliquots of the suspension plated out with 3 ml “Top agar” (38° C., P buffer to which 0.4% [m/V] low melting point agarose, Sigma, had been added) on regeneration medium R3.

For the selection of transformed protoplasts, antibiotics (sic) (50 μg/ml kanamycin) was added to the R3 medium. After drying the plates under the sterile workbench (approx. 30 min), the plates were incubated for 4-6 days at 37° C. The colony count was then determined.

No kanamycin-resistant transformants were obtained, although the regeneration of the protoplasts was successful, which could be demonstrated by a control experiment.

### Example 3 (not according to the invention)

Electroporation of Amycolatopsis sp. DSM 9992

A culture of Amycolatopsis sp. DSM 9992 in TYN medium (tryptone 10 g; yeast extract 2.5 g; NaCl 5 g; H₂O_{2x dist} to make up to 1000 ml; pH 7.2) at 150 rpm for approx. 20 h at 37° C. was used for the electroporation. The mycelium was harvested by centrifugation (4 500 rpm, 4° C., 15 min) and then washed twice with ice-cold salt-free water (Milli-Q Plus preparation system for highly pure water; MILLIPORE, Eschbom, Germany). The mycelium pellet was resuspended in 200 μl lysozyme solution (4 mg/ml; in 10% [V/V] glycerol) and incubated for 20 min at room temperature. The mycelium suspension was then washed with 10% [V/V] glycerol; centrifuging was carried out at 3 000 rpm and 4° C. for 10 min. The cells pretreated in this way were resuspended in a corresponding volume of glycerol (10% [V/V]) in such a way that a cell titre of approx. 1×10¹⁰ CFU/ml was obtained.

400 μl aliquots of the pretreated mycelium suspension were mixed with the plasmid DNA to be transferred (0.1-5.0 μg/μl) and transferred into cooled electroporation cuvettes (Eppendorf-Netheler-Hinz, Hamburg) with an electrode spacing of 2 mm. The electroporation was carried out at an electrical field strength of 7.5 kV/cm (capacitance 25 μF; resistance 600 Ω). Time constants of 3.6-5.6 ms were achieved. Immediately after the electroporation, 400 μl LB medium was added to the mycelium suspension. Immediately thereafter, 100 μl of the batch were plated out on GYM plates. After incubation for 15 h at 30° C. the plates were coated with 3 ml soft agar (TYN medium to which 5 g/l agar had been added), which contained antibiotics (for example 1000 μg/ml kanamycin) for the selection of positive electroporands. After drying the plates for 20 min, these were incubated for 3-5 days at 30° C. or 37° C. It was then possible to further investigate the possible positive electroporands obtained.

A transformation rate of 2×10² transformants per μg plasmid DNA was obtained for a field strength of 7.5 kV cm⁻¹ and a pulse of 4.6-5.2 ms.

### Example 4

Transformation of Amycolatopsis sp. DSM 9992 According to the Invention

The cells were cultured TSB medium. After 20 h growth (7 h stationary), the cell material was centrifuged off for 15 min at 4 500 rpm. After washing the harvested mycelium three times with TRIS-EDTA buffer, a dense mycelium suspension (OD_{400 nm}=50) was obtained by resuspension in a suitable volume of TE buffer. MgCl₂ (Merck Darmstadt, final concentration 5 mM), CsCl (ICN Biomedicals, final concentration 0.625 M), calf thymus DNA (Sigma-Aldrich, final concentration 37.5 ng/μl; stock solution 7.5 μg/ml), plasmid DNA (final concentration 1.25 μg/ml) and PEG 1 000 (NBS Biologicals, final concentration 32.5% [m/V]) were added to 100 μl of this suspension. The total volume of the transformation mixture was 400 μl. The plasmid DNA of the model vector pRLE6 that was used was isolated from E. coli ET12567 as described above in Example 1.

The transformation mixture was incubated for 40 min at 37° C. The cells were washed twice with 1 ml S27M medium in each case. The mycelium was resuspended in 400 μl S27M medium and incubated on ice for 10 min. Aliquots of the cells were then mixed with R2L agarose solution (temperature controlled to 42° C.) and applied to well-dried S27M agar plates. The batches were optionally diluted with S27M medium beforehand. After incubating for 16-20 h at 30° C., the selection was made by coating the plates with kanamycin-containing soft agar (S27M medium with 5 g/l agar) and subsequent incubation at 37° C. for 5-10 days.

The following examples were carried out analogously to Example 4, 1 parameter (see tables) being varied in each case.

TABLE 1
Dependence of the transformation
rate on the amount of DNA used

| DNA [μg] | Transformation rate | Transformation rate |
|---|---|---|
| per batch | [CFU/μg DNA] | [%] |
| 0 | 0 | 0 |
| 0.1 | 3.7 × 10⁵ | 97 |
| 0.25 | 3.5 × 10⁵ | 92 |
| 0.5 | 3.3 × 10⁵ | 87 |
| 1 | 3.7 × 10⁵ | 87 |
| 2 | 3.8 × 10⁵ | 100 |
| 3 | 3.7 × 10⁵ | 97 |

TABLE 2
Dependence of the transformation rate
on the PEG concentration used

| PEG concentration | Transformation rate | Transformation rate |
|---|---|---|
| [% (m/V)] | [CFU/μg DNA] | [%] |
| 25 | 4.0 × 10⁴ | 5.6 |
| 30 | 1.2 × 10⁵ | 17 |
| 32.5 | 7.2 × 10⁵ | 100 |
| 35 | 5.8 × 10⁵ | 81 |
| 40 | 4.2 × 10⁵ | 58 |

TABLE 3
Dependence of the transformation rate
on the CsCl concentration used

| CsCl | | |
|---|---|---|
| concentration | Transformation rate | Transformation rate |
| [M] | [CFU/μg DNA] | [%] |
| 0.0 | 0 | 0 |
| 0.1 | 1.1 × 10¹ | 0.002 |
| 0.2 | 5.5 × 10³ | 0.8 |
| 0.3 | 1.1 × 10⁵ | 16.2 |
| 0.4 | 1.6 × 10⁵ | 23.5 |
| 0.5 | 2.8 × 10⁵ | 41.2 |
| 0.6 | 6.8 × 10⁵ | 100 |
| 0.625 | 5.1 × 10⁵ | 75 |
| 0.7 | 2.1 × 10⁵ | 30.9 |
| 0.8 | 8.4 × 10⁴ | 12.4 |
| 0.9 | 4.0 × 10⁴ | 5.6 |
| 1.0 | 7.1 × 10⁴ | 10.4 |

TABLE 4
Dependence of the transformation rate
on the MgCl₂ concentration used

| MgCl₂ concentration | Transformation rate | Transformation rate |
|---|---|---|
| [mM] | [CFU/μg DNA] | [%] |
| 0 | 3.6 × 10⁵ | 52 |
| 2.5 | 4.9 × 10⁵ | 71 |
| 5 | 6.9 × 10⁵ | 100 |
| 7.5 | 3.9 × 10⁵ | 57 |
| 10 | 9.2 × 10⁴ | 13 |
| 15 | 8.5 × 10³ | 1.2 |
| 20 | 8.0 × 10² | 0.1 |

TABLE 5
Dependence of the transformation rate on the cell density of the
mycelium suspension used

| | Transformation rate | Transformation rate |
|---|---|---|
| OD₄₀₀ₙₘ | [CFU/μg DNA] | [%] |
| 5 | 2.4 × 10⁴ | 6.2 |
| 10 | 5.8 × 10⁴ | 14.9 |
| 20 | 6.4 × 10⁴ | 16.4 |
| 30 | 2.3 × 10⁵ | 59.0 |
| 40 | 3.5 × 10⁵ | 89.7 |
| 50 | 3.9 × 10⁵ | 100 |
| 60 | 3.5 × 10⁵ | 89.7 |
| 70 | 3.1 × 10⁵ | 79.5 |
| 80 | 2.4 × 10⁵ | 61.5 |
| 90 | 1.9 × 10⁵ | 48.7 |
| 100 | 1.8 × 10⁵ | 46.2 |
| 120 | 1.3 × 10⁵ | 33.3 |
| 160 | 1.1 × 10⁵ | 28.2 |

TABLE 6
Dependence of the transformation rate on the concentration of calf
thymus DNA (CT DNA) used

| Concentration | | |
|---|---|---|
| CT DNA | Transformation rate | Transformation rate |
| [ng/μl] | [CFU/μg DNA] | [%] |
| 0 | 1.4 × 10⁴ | 3.4 |
| 9.5 | 1.5 × 10⁵ | 36.6 |
| 14 | 2.4 × 10⁵ | 58.5 |
| 19 | 4.1 × 10⁵ | 100 |
| 28 | 3.1 × 10⁵ | 75.6 |
| 38 | 1.9 × 10⁵ | 46.3 |
| 47 | 2.0 × 10⁵ | 48.8 |
| 56 | 1.9 × 10⁵ | 46.3 |
| 75 | 1.0 × 10⁵ | 24.4 |
| 113 | 7.7 × 10⁴ | 18.8 |
| 150 | 5.5 × 10⁴ | 13.4 |
| 188 | 4.2 × 10⁴ | 10.2 |

TABLE 7
Dependence of the transformation rate on the
physiological status of the cells used
(Determination of the optical density in
Klett units was carried out using a Klett
colorimeter (Manostat Corp., USA)
at a wavelength of 520-580 nm)

| | | | |
|---|---|---|---|
| | Optical | | |
| | density | Transformation | Transformation |
| | at time of | rate | rate |
| Incubation period | harvesting | [CFU/μg DNA] | [%] |

(a)

| | | | |
|---|---|---|---|
| 8.5 h | 310 KU | 0 | 0 |
| 11 h | 570 KU | 1.3 × 10² | 0.08 |
| 15 h/approx. 2 h stationary | 590 KU | 1.3 × 10³ | 0.8 |
| 20 h/approx. 7 h stationary | 620 KU | 1.6 × 10⁵ | 100 |
| 40 h/approx. 27 h stationary | 570 KU | 0 | 0 |

(b)

| | | | |
|---|---|---|---|
| 18 h/approx. 5 h stationary | 620 KU | 8.0 × 10⁴ | 42 |
| 20 h/approx. 7 h stationary | 620 KU | 1.9 × 10⁵ | 100 |
| 24 h/approx. 11 h stationary | 610 KU | 2.0 × 10³ | 1 |
| 28 h/approx. 15 h stationary | 610 KU | 0 | 0 |

(c)

| | | | |
|---|---|---|---|
| 18 h/approx. 3 h stationary | 510 KU | 1.1 × 10⁵ | 15 |
| 20 h/approx. 5 h stationary | 490 KU | 2.2 × 10⁵ | 31 |
| 22 h/approx. 7 h stationary | 480 KU | 7.2 × 10⁵ | 100 |
| 24 h/approx. 9 h stationary | 450 KU | 2.2 × 10⁵ | 31 |

### Example 5

Transformation of Amycolatopsis sp. DSM 9992 According to the Invention

Example 5 was carried out analogously to Example 4, the mycelium being harvested 7 h after the stationary phase was reached (OD_{400 nm}=50). PEG 1000 was used in a final concentration of 32.5 5 (sic) [mNV], MgCl₂ in a final concentration of 5 mM, CsCl in a final concentration of 0.6 M and calf thymus DNA in a final concentration of 19 ng/μl; furthermore, 0.5 μg of the plasmid pRLE6 (isolated in accordance with Example 1) were (sic) used.

The example was repeated, the plasmids described in Table 8 being used instead of the plasmid pRLE6 isolated from E. coli ET12567. The plasmid isolated from E. coli XL1 Blue has a higher degree of methylation than the plasmid isolated from E. coli ET12567 and Amycolatopsis sp. DSM 9992.

| Origin of the plasmid | Transformation rate | Transformation rate |
|---|---|---|
| pRLE6 | [CFU/μg DNA] | [%] |
| E. coli ET12567 | 7.3 × 10⁵ | 100 |
| E. coli XL1 Blue | 2.1 × 10² | 0.03 |
| Amycolatopsis sp. DSM | 7.1 × 10⁵ | 97 |
| 9992 | | |

1. Method for the transformation of Amycolatopsis sp. DSM 9991 or DSM 9992 by
(a) culturing Amycolatopsis sp. DSM 9991- or DSM 9992 mycelia in a culture medium and
(b) bringing this culture into contact with a mixture containing
(i) 0.25 to 10 μg/ml DNA to be transformed
(ii) 0.4 to 0.7 M CsCl
(iii) 0to 9 mM MgCl₂
(iv) 30 to 50% [m/V] polyethylene glycol having an average molecular weight of 1000, and
(v) 10 to 50 μg/ml DNA which differs from (a), the culture being brought into contact with the said mixture 4.5 to 9 hours after formation of stationary mycelia cells.

2. Method according to claim 1, wherein the culture is brought into contact with the said mixture 5 to 8.5 hours after formation of stationary mycelia cells.

3. Method according to claim 1, wherein the said mixture contains 0.5 to 0.675 M CsCl.

4. Method according to claim 1, wherein the said mixture contains 2.5 to 7.5 mM MgCl₂.

5. Method according to claim 1, wherein the said mixture contains 12 to 30 μg/ml DNA which differs from (a).

6. Method according to claim 1, wherein (e) is calf thymus DNA.

7. Method according to claim 1, wherein the said mixture contains 32 to 35% (m/V) of said polyethylene glycol.

8. Method according to claim 1, wherein (a) is a DNA with a low degree of methylation.

9. Transformed Amycolatopsis sp. DSM 9991 or 9992, wherein the transformation has been carried out in accordance with a method according to claim 1.

10. Use of Amycolatopsis sp. DSM 9991 or 9992 according to claim 9 for the preparation of vanillin.

11. Use of Amycolatopsis sp. DSM 9991 or 9992 according to claim 9 for the preparation of vanillin from ferulic acid.

12. A method for the preparation of vanillin, characterised in that transformed Amycolatopsis sp. DSM 9991 or 9992 according to claim 9 is used.
